(19) United States
(12) Patent Application Publication
(10) Pub. No.: US 2010/0209910 A1
(43) Pub. Date: Aug. 19, 2010

(54) Methods and systems for identifying polynucleotide sequences with translational self-cleavage activity

(76) Inventors: Tzong-Yuan Wu, Panchiao City (TW); Chung-Hsiung Wang, Tainan City (TW); Yu-Jie Chen, Jhongli City (TW); Chao-Yi Teng, Jhongli City (TW); Ying-Ju Chen, Shinyuan Township (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/378,609

(22) Filed: Feb. 18, 2009

(51) Int. Cl. C12Q 1/68; C12N 5/06

(52) U.S. Cl. 435/6; 435/348

(57) ABSTRACT

Provided herein are methods and systems for identifying 2A-like sequences with translational self-cleavage activity in an insect expression system.

### SEQUENCE LISTING

The present disclosure includes a sequence listing incorporated herein by reference in its entirety.

### BACKGROUND

1. Field of Invention

This invention in general relates to a method and/or a system for identifying an isolated polynucleotide with a translational self-cleavage activity. Specifically, this invention relates to a method and/or a system for identifying a 2A-like sequence by use of a fluorescence distribution pattern in an insect expression system.

2. Description of Related Art

A bi-cistronic or multi-cistronic expression vector is useful in various aspects, such as heterologous oligomeric proteins production, gene therapy, cellular tissue engineering and etc., allowing the equimolar expression of the target proteins.

Current strategies for creating multi-cistronic vectors include the use of internal ribosome entry sites (IRES), multiple promoters, and fusion proteins with or without linkages via cellular protease sites. However, these three strategies have encountered respective obstacles in their practical application.

IRES elements can be large, and thus their use might be confined in size-restricted vectors (e.g., viral and transposon-based vectors). Furthermore, downstream gene expression would be attenuated potentially due to the order of the genes on the transcript (Hennecke, M., et al., (2001) Nucleic Acids Res. 29: 3327-3334; Fux, C. et al., (2004) Biotechnol. Bioeng. 86: 174-87).

Fusion protein production may result in compromised function potentially due to improper protein folding or trafficking. And, the use of multiple promoters results in less intimate linkage between expression of reporter gene and that of target gene. (Gaken, J., et al., (2000) Gene Ther. 7: 1979-1985; de Felipe, P., (2004) Genet. Vacc. Ther. 2: 1-12).

Recent strategies involve the use of the foot-and-mouth disease virus (FMDV) 2A or 2A-like cis-acting hydrolase elements (CHYSEL) to create multicistronic vectors capable of generating multiple proteins from the same transcript (Szymczak, A. L., et al., (2004) Nat. Biotechnol. 22: 589-594; de Felipe, P., et al., (1999) Gene Ther. 6: 198-208; El Amrani, A., et al., (2004) Plant Physiol. 135: 16-24). Many viruses encode multiple proteins that are cleaved into individual protein products at 2A or 2A-like sequences (Palmenberg, A. C., et al., (1992) Virology 190: 754-762; Donnelly, M. L. L., et al., (2001) J. Gen. Virol. 82: 1027-1041; Ryan, M. D. et al., (1991) J. Gen. Virol. 72: 2727-2732). The 2A-like sequence contains a canonical Asp-Val/Ile-Glu-X-Asn-Pro-Gly-Pro motif, where the linkage between the glycine and the proline residues would be self-cleaved (Palmenberg, A. C. (1990) Annu. Rev. Microbiol. 44: 603-623; Hahn, H. and Palmenberg, A. C., (1996) J. Virol. 70: 6870-6875). The cleavage mechanism might result from ribosomal skipping mechanism where ribosome activity is modified by the 2A-like sequences so as to prevent peptide bond formation between the Glycine and the Proline residues. Therefore, the upstream protein of the 2A-like sequence will be released while allowing continued translation of the downstream gene (de Felipe, P. et al., (2003) J. Biol. Chem. 278: 11441-11448; Donnelly, M. L. L., et al., (2001) J. Gen. Virol. 82: 1013 - 1025).

Most of the 2A-like sequences are found in viruses that infected mammalian cells. However, only few 2A-like sequences are cloned from insect viruses. Furthermore, the current known virus 2A-like sequences still exhibit incomplete self-cleavage during mRNA translation in an insect expression system, resulting in insufficient amount of the respective upstream and downstream target peptides or proteins.

Accordingly, there remains a need of search an improved 2A-like sequences capable of completely self-cleaved, so that the yield of the respectively encoded upstream and downstream protein operably linked thereto are enhanced. This invention addresses the aforementioned problem by providing an efficient and easy-to-use method and/or system for identifying potential 2A-like sequences by use of distinctive fluorescence distribution patterns exhibited in an insect expression system as indicia for determining whether the potential sequences are the desired 2A-like sequences or not.

### SUMMARY

As embodied and broadly described herein, the invention features methods and/or systems for identifying polynucleotide sequences that are capable of inducing ribosomal skipping during mRNA translation in an insect expression system.

Therefore, it is an objective of this invention to provide a method of identifying a 2A-like sequence in an insect system. The method is characterized in having the steps of:

a) infecting an insect cell with a recombinant vector comprising:
a promoter;
a first polynucleotide operably linked to the promoter and encodes a secretory protein;
a candidate sequence operably linked to the first polynucleotide; and
a second polynucleotide operably linked to the candidate sequence and encodes a fluorescent protein; and

b) determining whether the candidate sequence possesses the translational self-cleavage activity based on a fluorescence distribution pattern in the insect cell, if the fluorescence is distributed homogenously in the insect cells Is including the nucleus, then the candidate sequence is the 2A-like sequence; if the fluorescence is limited to extra-nucleus space or is secreted to extracellular medium, then the candidate sequence is not the 2A-like sequence.

In one preferred example, the candidate sequence is isolated from Perina nuda Picorna -like virus (PnV) and comprises a polynucleotide sequence of SEQ ID NO: 1. The sequence identified by the method of this invention cencodes a polypeptide with an amino acid sequence of SEQ ID NO: 2, which includes a transitional self-cleavage site located between Glycine and Proline, and has a translational self-cleavage activity up to 100%, indicating that the secortory protein and the fluorescent protein are produced in a ratio of about 1:1 in vivo. In another preferred example, the candidate sequence is isolated from Perina nuda Picorna-like virus (PnV) and comprises a polynucleotide sequence of SEQ ID NO: 3, which cencodes a polypeptide with an amino acid sequence of SEQ ID NO: 4. In one example, the secretory protein is secretory alkaline phosphatase (SEAP); and the fluorescent protein is an enhanced green fluorescence protein (EGFP).

It is another objective of this invention to provide an insect system for identifying a 2A-like sequence having a translational self-cleavage activity. The system comprises:

an insect cell infected with a recombinant vector comprising:
a promoter;
a first polynucleotide operably linked to the promoter and encodes a secretory protein;
a candidate sequence operably linked to the first polynucleotide; and
a second polynucleotide operably linked to the candidate sequence and encodes a fluorescent protein;
   wherein a fluorescence distribution pattern in the insect cell when observed under a fluorescence microscope is used as an indicator to determine whether the candidate sequence is the 2A-like sequence, if the fluorescence is distributed homogenously in the insect cells including the nucleus, then the candidate sequence is the 2A-like sequence; if the fluorescence distribution pattern has a donut shape and is limited to extra-nucleus space or is secreted to extracellular medium, then the candidate sequence is not the 2A-like sequence.

The details of one or more embodiments of the invention are set forth in the accompanying description and drawings below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The invention will be illustrated with respect to the accompanying figures and examples, which serve to illustrate this invention but are not binding thereon, wherein:

FIG. 1 illustrates a schematic diagram of the recombinant vectors of (a) vAc-S-Rhir-E, (b) vAc-SEFP and (c) (vAc-S-PnV2A1-E);

FIG. 2 are fluorescent photographs of Sf21 insect cells taken on day 4 after being infected with the recombinant vectors of FIG. 1;

FIG. 3 is a Western blot diagram illustrating the respective expression levels of EGFP genes in Sf21 cell infected with the recombinant vectors of FIG. 1;

FIG. 4 is a bar diagram illustrating the respective SEAP activities in the Sf21 insect cells infected with the recombinant vectors of FIG. 1; and

FIG. 5 is a bar diagram illustrating the quantification result of EGFP expression in Sf21 insect cells infected with the recombinant vectors of FIG. 1.

### DETAILED DESCRIPTION

Unless otherwise indicated, all terms used herein have the same meaning as they would to one skilled in the art and the practice of this invention will employ conventional techniques of microbiology and recombinant DNA technology, which are within the knowledge of those of skill of the art.

The practices of this invention are hereinafter described in detail with respect to systems and/or methods for the identification of polynucleotides having translational self-cleavage activities.

According to one embodiment of this invention, a method for identifying a 2A-like sequence in an insect system is provided but not limited to the insect cell system demonstrated herein. The method is characterized in having the steps of: (a) infecting an insect cell with a recombinant vector comprising: a promoter; a first polynucleotide operably linked to the promoter and encodes a secretory protein; a 2A-like candidate sequence operably linked to the first polynucleotide; and a second polynucleotide operably linked to the candidate sequence which encodes a fluorescent protein; and (b) determining whether the 2A-like candidate sequence possesses a translational self-cleavage activity based on a fluorescence distribution pattern in the insect cell, if the fluorescence is distributed homogenously in the insect cells including the nucleus, then the candidate sequence is the 2A-like sequence; if the fluorescence distribution pattern has a donut shape and is limited to extra-nucleus space or is secreted to extracellular medium, then the candidate sequence is not the 2A-like sequence.

For the present invention, 2A-like sequence having translational self-cleavage activity is a DNA or cDNA derived from a virus, preferably, from Picornaviridae family, including but not limited to enterovirus, rhinovirus, hepatovirus, cardiovirus, aphthovirus, parechovirus, erbovirus, kobuvirus, parechovirus, teschovirus or the insect derived Picorna-like virus and iflavirus. The term “translational self-clevage activity” is defined herein as a translational effect of a sequence capable of auto-proteolysis or cleavage by cleaving its own C-terminus to produce primary cleavage products. The potential 2A-like sequences or candidate sequences may be selected in accordance with the sequence homology, the secondary or tertiary structure similarity, or the existence of conserved regions corresponding to any known 2A sequence, such as 2A sequences found in cardioviral and aphthoviral genome, for example, a 2A sequence of Foot-and-mouth disease virus (FMDV). In one preferred embodiment, the candidate sequence is isolated from Perina nuda Picorna-like virus (PnV) based on sequence homology, and comprises a polynucleotide sequence of SEQ ID NO: 1 or 3, which is at least 80%, 81%, 82%, 83%, 84%, 85%, 86%, 87%, 88%, 89%, 90%, 99%, 91%, 92%, 93%, 94%, 95%, 96%, 97%, 98%, 99% or 100% identical to any known 2A sequence.

The candidate sequence selected in accordance with the manner described above is then cloned into a recombinant viral vector with two reporter genes. By “reporter gene” herein is meant a gene that by its unique characteristics such as fluorescence or secretion ability in a host cell (i.e., upon expression) allows the selected candidated sequences to be identified in accordance with the criteria specified in this invention, which are further described below. In the present invention, expression of the reporter genes causes the effects, such as fluorescence distribution pattern in the host cells, to be used, as an indicative for easy determination of the candidate sequence as a desired 2A-like sequence under a fluorescence microscope. In one preferred example, the recombinant viral vector is engineered to contain in sequence: a promoter; a first reporter gene operably linked to the promoter and encodes a secretory protein; the candidate sequence operably linked to the first reporter gene; and a second reporter gene operably linked to the candidate sequence and encodes a fluorescent protein. By “promoter” herein is meant nucleic acid sequence capable of initiating transcription of the coding sequence downstream of the promoter sequence. Promoters may be inducible or cell specific. Preferred promoters for expression in an insect cell system are polyhedrin, p10, vp39, ie1 and ie2 promoters, most preferably, a polyhederin promoter. The term “operably linked” is meant that a polypeptide coding sequence and transcriptional and translational controlling sequences in a recombinant vector are connected in such a way as to permit polypeptide expression when appropriate molecules (e.g., transcriptional activation proteins) are bound to the regulatory sequence. The first reporter gene encodes a secretory protein, such as secreted human placenta alkaline phosphatase (SEAP). The second reporter gene encodes a fluorescent protein, including but not limited to, green fluorescent protein, (GFP), enhanced green fluorescent protein (EGFP), blue fluorescent protein (BFP), enhanced yellow fluorescent protein (EYFP), Anemonia majano fluorescent protein (amFP), Zoanthus fluorescent protein (zFP), Discosoma fluorescent protein (dsFP), Clavularia fluorescent protein (cFP), luciferase (such as firefly luciferase), Renilla reniformis luciferase, and Renilla muelleri luciferase. In one example, the first reporter gene encodes SEAP, and the second reporter gene encodes EGFP. Suitable viral vectors are those from baculoviruses, including but not limited to, Autographa californica nuclear polyhedrosis virus (AcMNPV), PnMNPV (Perina nuda multinucleocapsid nuclear polyhedrosis virus), BmNPV (Bombyx mori nuclear polyhedrosis virus), LdMNPV (Lymantria dispar multinucleocapsid nuclear polyhedrosis virus) and OpMNPV (Orgyia pseudotsugata multicapsid nucleopolyhedrovirus).

The recombinant vector prepared in accordance with a preferred example of this invention is subsequently used to transfect an insect expression system, such as S. frugiperda IPBL-Sf21 insect cell line. However, other insect expression system may be used as well. As will be appreciated by those skilled in the art, if the candidate sequence incorporated into the aforementioned fusion nucleic acid (i.e., the nucleic acid comprises in sequence: a first reporter gene, a candidate sequence and a second reporter gene) is a 2A-like sequence, it will lead to expression of separate protein products of the first and second reporter gene, i.e., the secretory protein and the fluorescent protein, and the secretory protein is eventually transported to extracellular medium whereas the fluorescent protein would remain inside the host cells. In contrast, if the candidate sequence incorporated into the fusion nucleic acid is not a desired 2A-like sequence, the expressed candidate sequence will act as a peptide linker to link the first and second protein products together and generate a fused protein. The fused protein, or the two reporter proteins fused together, is subsequently transported out of the host cell due to the secretory portion of the fused protein. Therefore, by simply observing the fluorescence distribution pattern of the host cell under a fluorescent microscope, provides an easy and efficient means for determining whether the candidate sequence is a 2A-like sequence or not, instead of reverting to more traditional, time-consuming manners of determination such as Western blot assay or protein activity measurement. In one preferred example, a homogeneous fluorescence pattern in the host cell indicates that the candidate sequence is a desired 2A-like sequence, which is capable of self-cleavage to produce two protein products. In another example, a donut shape fluorescence pattern is observed, indicates that the candidate sequence is not a desired 2A-like sequence, and a fused protein, instead of two separate protein products, is produced and secreted out of the host cell; therefore, fluorescence is limited to extra-nucleus space of cytosol or is secreted to extracellular medium and thereby forming a donut shape fluorescence pattern.

In one preferred example, the identified 2A-like sequence or the candidate lo sequence comprises a polynucleotide sequence of SEQ ID NO:1, which encodes a polypeptide having an amino acid sequence of SEQ ID NO:2, with a conserved self-cleavage site located between Glycine and Proline residues. In another preferred example, the identified 2A-like sequence or the candidate sequence comprises a polynucleotide sequence of SEQ ID NO:3, which encodes a polypeptide having an amino acid sequence of SEQ ID NO:4,

Furthermore, the method and/or system of this invention provide a balanced or equimolecular production of the two reporter proteins, that is, the two reporter proteins are produced in relatively same proportion. In one preferred example, the secretory protein encoded by the first reporter gene and the fluorescent protein encoded by the second reporter gene are produced in a ratio of about 1:1.

According to another embodiment of this invention, a system for identifying a 2A-like sequence having a translational self-cleavage activity is provided. The system comprises: an insect cell infected with a recombinant vector comprising in sequence: a promoter; a first polynucleotide operably linked to the promoter and encodes a secretory protein; a candidate sequence operably linked to the first polynucleotide; and a second polynucleotide operably linked to the candidate sequence and encodes a fluorescent protein; wherein a fluorescence distribution pattern in the insect cell is used as an indicator to determine whether the candidate sequence is the 2A-like sequence, if the fluorescence is distributed homogenously in the the insect cells including the nucleus, then the candidate sequence is the 2A-like sequence; if the fluorescence distribution pattern has a donut shape, then the candidate sequence is not the 2A-like sequence.

To provide those skilled in the art the tools to use the present invention, the nucleic acids of the reporter genes and insect cells of the invention are assembled into kits. The components included in the kits are viral vectors, enzymatic agents for making the recombinant viral constructs, cells for amplification of the viruses, and reagents for transfection and transduction into the target cells, as well as description in a form of pamphlet, tape, CD, VCD or DVD on how to use the kits.

The following examples are provided to illustrate the present invention without, however, limiting the same thereto.

### EXAMPLE

### Building Plasmid Constructs

1.1 pBac-S-Rhir-E

The pBac-S-Rhir-E vector was constructed in accordance with a similar procedure described by Chen et al for building pBac-DRhir-E vector (Biochem. and Biophy. Res. Commu. 2005 335; 616-623) except a SEAP reporter gene was used in lieu of DsRed gene. Hence, the pBac-S-Rhir-E contains in sequence the SEAP reporter gene, the IRES sequence of Rhopalosiphum padi virus (RhPV) and the EGFP reporter gene.

1.2 pBac-SEFP

The SEAP gene was inframed-fused with the EGFP gene in the pBac-DRhir-E plasmid (Chen et al., Biochem. and Biophy.l Res. Commu. 2005 335; 616-623). Briefly, the SEAP fragment was first amplified from the pGS-HCV plasmid (Lee et al., Biotechno. and Bioeng. 2005 90:656-672) by polymerase chain reaction (PCR) with the forward primer, 5′-ATATAAGATCTCCACCATGCTGCTGCTGTGCTGCTGCTGGG-3′ (SEQ ID NO:5, with the Ba/II site underlined), and the reverse primer, 5′-AATTCAGATCTGGTGTCTGCTCGAAGCGGCCGGC-3′ (SEQ ID NO:6, with the Bg/II site underlined). Two GG nucleotides in the reverse primer (bold and italicized) were added to allow in-frame fusion between the 3′ end of the SEAP and the 5′ end of the EGFP gene. After PCR, the 1.6-kb, Bg/II -digested SEAP gene fragment was subcloned into the BamHI-digested pBac-DRhirE plasmid, and the resulting plasmid was named pBac-DRhir-SEFP, which contained in sequence the DsRed reporter gene, RhPV IRES sequence and the fusion gene of SEAP and EGFP.

The pBac-S-Rhir-E plasmid was then digested with BamHI and Sa/I restriction enzymes to remove the SEAP-Rhir-EGFP fragment. The pBac-DRhir-SEFP plasmid was also digested with BamHI and Sa/I to remove the fusion fragment of SEAP-EGFP. Then, the SEAP-EGFP fragment was cloned into the BamHI and Sa/I site of the pBac-S-Rhir-E plasmid, and the resulting plasmid is named pBac-SEFP.

### 1.3 PBac-S-PnV2Al-E

The SEAP-PnV2Al gene fragment was first amplified from the pBac-DsRed-PnV-SEAP-RhPV-EGFP plasmid by polymersae chain reaction (PCR) with the forward primer, 5′-AATGGATCCGCTAGCCCACCATGCTGCTGCTGCTGCTG-3′ (SEQ ID NO:7, with the BamHI site underlined) and the reverse primer, 5′-AATAGATCTAAGGGTCCGGGGATTTGACTCAACATCTCCATCCACAGTCAAA TCCGGMCCCACCCCTGGGCTGTCTGCTCGMGCGGCC-3′ (SEQ ID NO: 8, with the Bg/II site underlined).

The amplified SEAP-PnV2Al fragment and PCR2.1 (Invitrogen, U.S.A) were used for TA cloning, and the resulting plasmid is named PCR2.1/SEAP-PnV2Al hereafter. The PCR2.1/SEAP-PnV2Al plasmid was digested with BamHI and Bg/II to remove the SEAP-PnV2Al fragment. The removed SEAP-PnV2Al fragment was then ligated to the BamHI and Bg/II site of the pBac-S-Rhir-E plasmid, and the resulting plasmid is named pBac-S-PnV2Al-E hereafter.

### 1.4 Production of Recombinant Vectors

S. frugiperda IPBL-Sf21 insect cell line (hereinafter “Sf21 cells”) was cultured in TNM-FH medium containing 8% heat-inactivated FBS until a confluent cell monolayer (about 2×10⁵ cells/well) was obtained. The plasmid (0.8 μg) of example 1.1 (pBac-S-Rhir-E), 1.2 (pBac-SEFP) or 1.3 (pBac-S-PnV2Al-E) was transfected into the confluent Sf21 insect cells together with the linearized Bac-N-Blue baculovirus DNA (0.25 μg) by use of 1 μl of Cellfectin™ Transfection Reagent (Invitrogen Corp., Carlsbad, Calif.). The transfected cells were cultured in TNM-FH medium free of FBS for 12 hours, and subsequently cultured in TNM-FH medium containing 8% heat-inactivated FBS.

If DNA homologous recombination occurred among the Bac-N-Blue baculovirus DNA and the three vectors, that is, example 1.1 (i.e., pBac-S-Rhir-E), 1.2 (i.e., pBac-SEFP) and 1.3 (i.e., pBac-S-PnV2Al-E), new recombinant baculovirus expression vectors are formed, and these newly formed recombinant vectors could be selected by well known end-point dilution method with the aid of the green fluorescence emitted from the co-expressed EGFP. The selected recombinant baculovirus expression vectors were thus named vAc-S-Rhir-E, vAc-SEFP and vAc-S-PnV2Al-E, respectively.

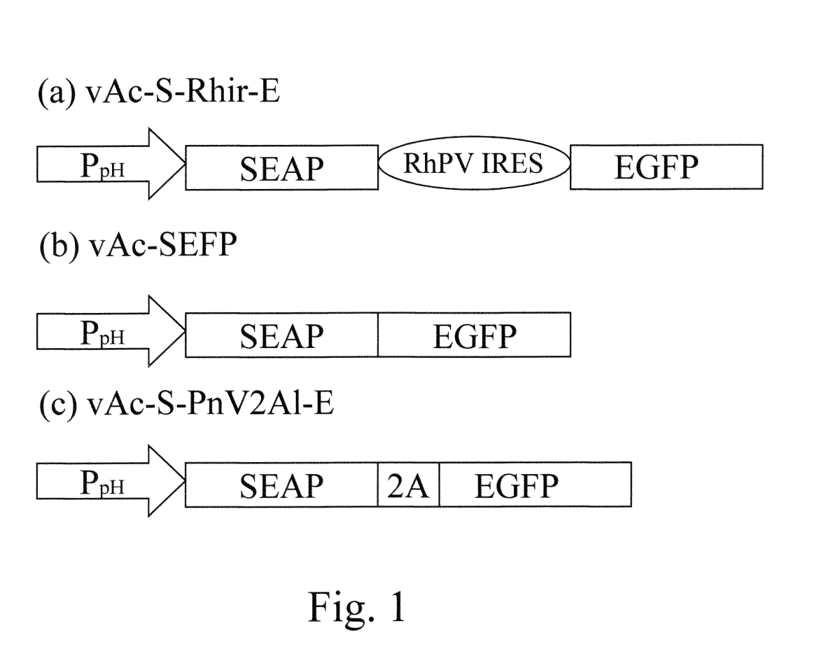

FIG. 1 is the schematic diagram of the respective recombinant virus expression vectors, including (A) vAc-S-Rhir-E, (B) vAc-SEFP and (C) vAc-S-PnV2Al-E. The promoter used in this invention is the polyhedron promoter (hereafter named as P_{PH}). The recombinant vAc-S-Rhir-E and vAc-SEFP virus expression vectors were used as a comparison to the vAc-S-PnV2Al-E vector.

### EXAMPLE 2

### Identifying 2A-Like Sequence

### 2.1 Selection Based on Fluorescence Pattern

If the selected PnV2A-like sequence in the constructed recombinant vectors of Example 1.4 did not possess ribosomal skipping activity, then the SEAP gene, the PnV2A-like sequence and the EGFP gene would be expressed in the same transcript. Hence, a fusion protein, containing in sequence SEAP, the PnV2A-like polypeptide and EGFP, would be expressed and secreted outside the cell, and the green fluorescence emitted from EGFP would be limited to the extra-nucleus space and looks like a green donut. On the contrary, if the selected PnV2A-like sequence did possess ribosomal skipping activity, then the PnV2A-like sequence would be self-cleaved. Hence, SEFP and EGFP would be expressed as independent polypeptides and EGFP would be distributed homogenously within the cell, including nucleus, so that the green fluorescence emitted from EGFP would be equally distributed in the cell.

The Sf21 cells (2×10⁵/well in a 24-well plate) respectively infected with the recombinant vectors vAc-S-Rhir-E, vAc-SEFP and vAc-S-PnV2Al-E, were lysed with 300 ml culture cell lysing reagent (100 mM potassium phosphate (pH 7.8), 1 mM EDTA, 10% Triton X-100, and 7 mM β-mercaptoethanol) for 10 min. After centrifugation at 12,800 rpm for 30 min, a small portion of the lysed supernatant (100 μl) was taken out for fluorescence measurement. The fluorescence spectrum of EGFP was measured using a Cary Eclipse fluorescence spectrophotometer (Varian) with excitation and emission wavelength set at 488 nm and 507 nm, respectively.

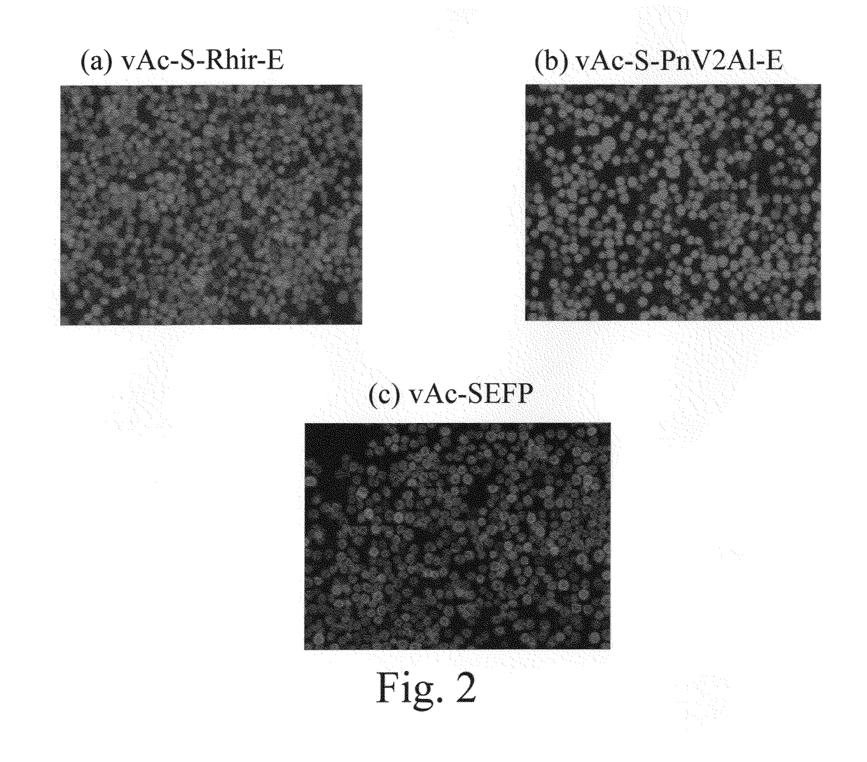

FIG. 2 illustrates the green fluorescence pattern in Sf21 cells respectively infected with the recombinant vectors of (A) vAc-S-Rhir-E, (B) vAc-S-PnV2Al-E and (C) vAc-SEFP. In FIG. 2A, it was inferred that the SEAP in Sf21 cells infected with vAc-S-Rhir-E would be expressed by the cap-dependent translation and the EGFP thereof would be expressed by RhPV IRES sequence according to Teng and Wu (2007), hence the green fluorescence emitted from EGFP would be homogeneous distributed in the cells, including the nucleus. In FIG. 2B, the green fluorescence pattern exhibited from Sf21 cells infected with vAc-S-PnV2A-E were similar to that of vAc-S-Rhir-E, indicating that the PnV2A-like sequence is capable of being self-cleaved and thereby the co-expressed EGFP is equally distributed in the nucleus.

On the contrary, in FIG. 2C, the green fluorescence in Sf21 cells infected with vAc-SEFP exhibited a donut shape pattern, indicating that a fusion protein (SEAP plus EGFP) was synthesized and secreted into the medium and therefore the fluorescence was limited to extranucleus space. In other words, if the selected 2A-like sequence in the baculovirus expression system exhibits a similar green fluorescence pattern as that of vAc-SEFP vector, then the selected 2A-like sequence is not a desired 2A-like sequence and is incapable of self-cleaved or inducing ribosomal skipping.

### 2.2 Selection Based on Western Blot Results

The self-cleaved translational activity of the selected PnV2A-like sequence was further confirmed by Western blot analysis. Briefly, the Sf21 cells respectively infected with vectors of examples 1.4 including vAc-S-PnV2Al-E, vAc-S-Rhir-E and vAc-SEFP were harvested and lysed. The proteins in the cell lysates were separated by SDS-PAGE and subsequently transferred onto a polyvinyidiene difluoride membrane (PVDF, Millipore). The membrane was then blocked with a Tris-buffer (100 mM Tris, pH7.4; 100 mM NaCl and 0.1% Tween 20) containing 5% bovine serum albumin (BSA, Sigma) at room temperature for 1 hour.

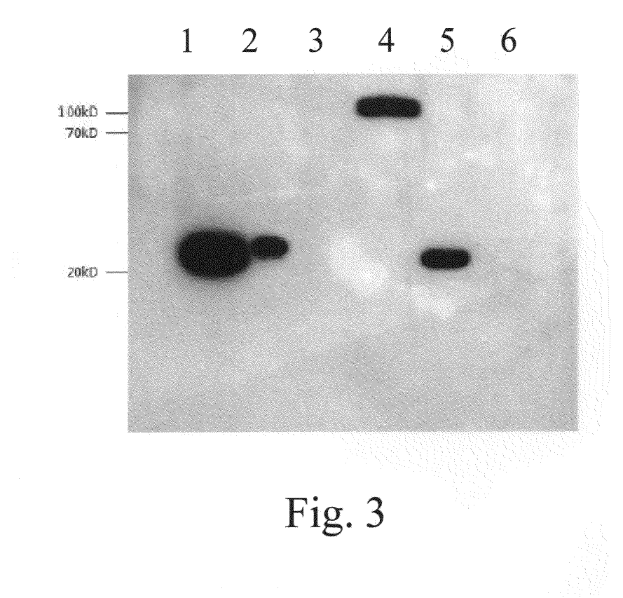

After blocking, the membrane was incubated with an anti-EGFP antibody (1:2000, BD Biosciences ClonTech, Palo Alto, Calif.) at 4° C. overnight. The membrane was then washed with Tris-buffer at room temperature three times, each time for 5 minutes, to remove the non-bonded anti-EGFP antibody. The PVDF membrane was then incubated with horseradish peroxidase (HRP) conjugated secondary antibody (1:2500) at room temperature for 1 hour. The PVDF membrane was washed with the same Tris-buffer at room temperature three times, each time for 5 minutes to remove any non-bonded secondary antibody. Results were provided in FIG. 3.

If the PnV2A-like sequence successfully induced ribosomal skipping, the EGFP protein and SEAP protein would be expressed independently, and a EGFP protein of approximately 27 kDa would be detected. In contrast, if the transcript of the PnV2A-like sequence were not self-cleaved, a fusion protein of EGFP and SEAP with molecular weight around 100 kDa would be produced

FIG. 3 illustrates the respective EGFP levels of the SF21 insect cell infected with the recombinant vectors vAc-S-PnV2Al-E, vAc-SEFP and vAc-S-Rhir-E in cytosol (within the cell) (lanes 1, 3 and 5, respectively) and medium (secreted protein) (lanes 2, 4 and 6, respectively). It is clear that the EGFP of about 27 kDa was produced so that the selected PnV2A-like sequence in vAc-S-PnV2Al-E is capable of being self-cleaved (lane 1 vs lane 2). vAc-S-Rhir-E also exhibited similar pattern (lane 5 vs lane 6) as that of vAc-S-PnV2Al-E. In lane 4, a EGFP protein of about 100 kDa was detected, indicating that the EGFP was fused with SEAP.

Moreover, the result illustrated in lanes 1 and 2 of FIG. 3 further indicated that the cleavage efficiency of the PnV2Al-like sequence can be up to 100%, for the amount of fusion protein (˜100 kDa molecular weight) is negligible.

### 2.3 Selection Based on Secretory Alkaline Phosphatase (SEAP) Activity

The cleavage efficiency of the identified PnV2A-like sequence can be further clarified by the measurement of SEAP activity.

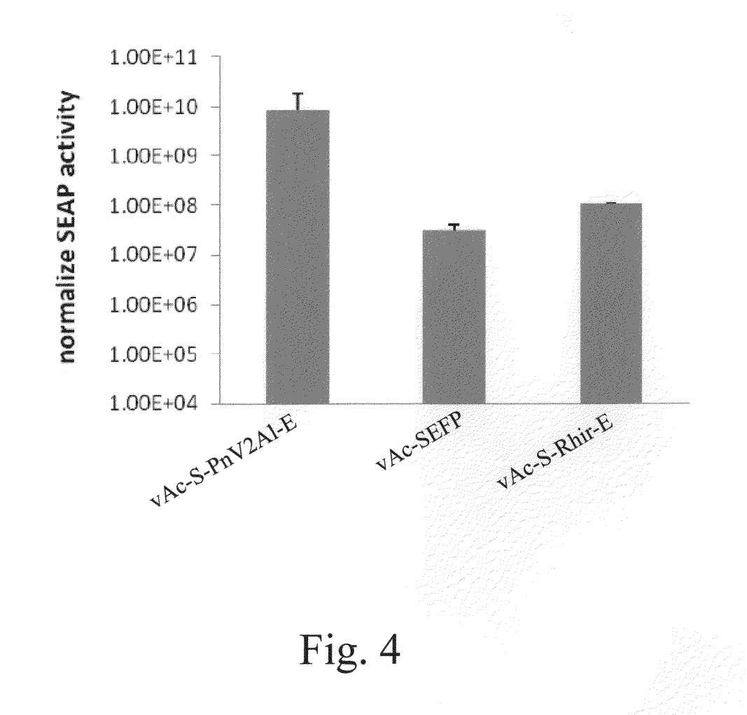

The medium of Sf21 cells (2×10⁵/well in a 24-well plate) infected with vAc-S-PnV2Al-E, vAc-SEFP and vAc-S-Rhir-E were collected respectively and centrifuged at a speed of 12,000 g for 10 seconds and then held at −20° C. until the SEAP activity assay was performed. The SEAP activity in culture medium was measured using a BD Great EscApe SEAP detection kit (Clontech). The chemiluminescent intensities reflecting relative SEAP activities were detected with a chemical luminescence counter (Mithras LB 940). Result was provided in FIG. 4.

FIG. 4 illustrates the different SEAP activities of the SF21 insect cells respectively infected with the viral vector of vAc-S-Rhir-E, vAc-SEFP and vAc-S-PnV2Al-E. It is clear that the SEAP activity of the Sf21 insect cell infected with the vAc-S-PnV2Al-E (MOI=5, dpi=4) is much higher than that infected with the vAc-SEFP, approximately five hundreds folds. Hence, the selected PnV2A-like sequence in the recombinant vAc-S-PnV2Al-E can successfully induce ribosomal skipping so that the interference defect of protein folding within the fusion protein is avoided, and the activity of the target protein is unaffected. Interestingly, the SEAP activity of the Sf21 insect cell infected with the vAc-S-PnV2Al-E is also much higher than that infected with the vAc-S-Rhir-E, where both SAEP and EGFP protein are also separately expressed. The SEAP activity of the SF21 insect cell infected with the vAc-S-PnV2Al-E is approximately one hundred fold higher than that infected with the vAc-S-Rhir-E. Inventors believe that this is probably due to the fact that the mRNA of the PnV2A-like sequence is more stable than that of RhPV IRES sequence.

### 2.4 Green Fluorescence Quantification

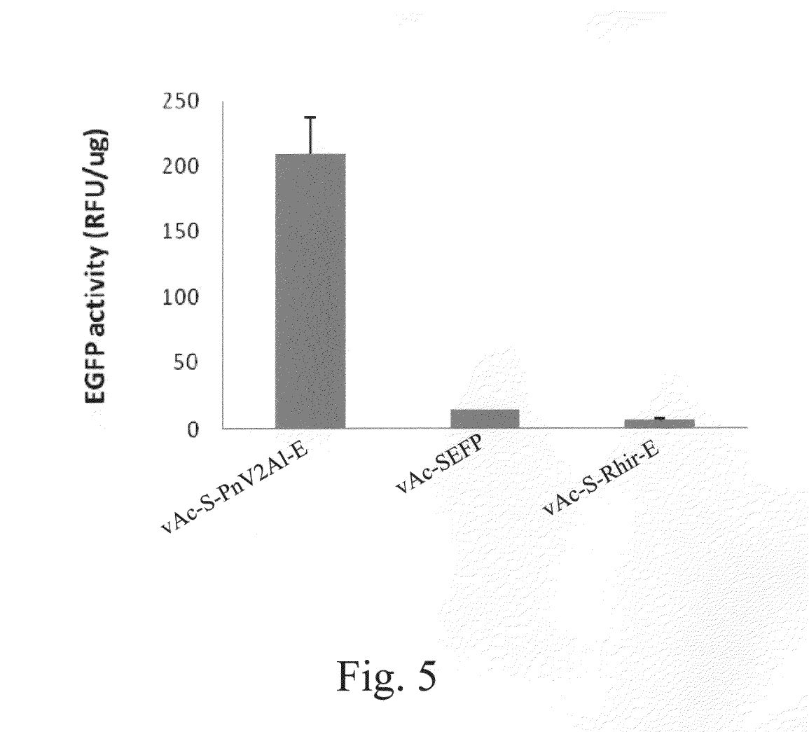

The EGFP protein on the PVDF membrane was detected by Enhanced Chemiluminescence Kit (Piece) to quantify the expressed amount of EGFP. Result was provided in FIG. 5, which illustrated the green fluorescence intensities of the SF21 cells respectively infected with the vAc-S-PnV2Al-E, vAc-SEFP and vAc-S-Rhir-E. Again, the fluorescence intensity of EGFP in the SF21 insect cell infected with the vAc-S-PnV2Al-E is much higher than those infected with the vAc-SEFP and the vAc-S-Rhir-E, respectively. Hence, it is clear that the self-cleaved part of the PnV2A-like sequence fused with EGFP does not affect the fluorescence intensity of EGFP.

### EXAMPLE 3

### Another Embodiment of a 2A-Like Sequence Identified by the Method of Example 2

Another 2A-like sequence was cloned and identified in accordance with the procedures described in Example 2. The transfected insect host cells exhibited a donut like fluorescence pattern (data not shown) and therefore confirmed the candidate sequence to be a desired 2A-like sequence. The sequence thus identified comprises a polynucleotide sequence of SEQ ID NO:3, which encodes a polypeptide having an amino acid sequence of SEQ ID NO:4.

### INDUSTRIAL APPLICABILITY

This invention takes advantage of a sectory protein and a fluorescent protein co-expressed in an insect host cell as reporter proteins for screening potential 2A-like sequences. Insect host cells are first transfected with a vector comprising a candidate sequence and the two reporter genes engineered in accordance with the method of this invention, and the decision of whether the candidate sequence is a desired 2A-like sequence was determined by simply viewing the transformed host cells under a fluorescent microscope. A donut shape fluorescent patterns indicates that the selected candidate sequence is not a 2A-like sequence; whereas a homogeneous distributed fluorescent pattern indicates that the selected candidate sequence is a desired 2A-like sequence. Therefore, this invention provides a fast and easy-to-use screening method and/or system for identifying polynucleotides having translational self-cleavage activity, without the hassle of performing more labor-intensive and time-consuming protein measurements such as Western blot.

While the foregoing is directed to embodiments of this invention, other and further embodiments of the invention may be devised without departing from the basic scope thereof, and the scope thereof is determined by the claims that follow.

1-8. (canceled)

9. An insect system for identifying a 2A-like sequence having a translational self-cleavage activity, comprising:
an insect cell infected with a recombinant vector comprising in sequence:
a promoter;
a first polynucleotide operably linked to the promoter and encodes a secretory protein;
a candidate sequence operably linked to the first polynucleotide; and
a second polynucleotide operably linked to the candidate sequence and encodes a fluorescent protein;
wherein a fluorescence distribution pattern in the insect cell is used as an indicator to determine whether the candidate sequence is the 2A-like sequence; if the fluorescence is distributed homogenously in the insect cell including the nucleus, then the candidate sequence is the 2A-like sequence; if the fluorescence distribution pattern has a donut shape and is limited to extra-nucleus space or is secreted to extracellular medium, then the candidate sequence is not the 2A-like sequence.

10. The system of claim 9, wherein the candidate sequence is isolated from Perina nuda Picorna-like virus (PnV) and comprises a polynucleotide sequence of SEQ ID NO: 1 or 3.

11. The system of claim 10, wherein the candidate sequence encodes a polypeptide comprising an amion acid sequence of SEQ ID NO: 2 or 4, which includes a transitional self-cleavage site located between Glycine and Proline.

12. The system of claim 11, wherein the translational self-cleavage activity of the candidate sequence is up to 100%.

13. The system of claim 12, wherein the secretory protein and the fluorescent protein is produced in a ratio of about 1:1

14. The system of claim 9, wherein the insect cell is a S. frugiperda IPBL-Sf21 insect cell.

15. The system of claim 9, wherein the fluorescent protein is an enhanced green fluorescence protein (EGFP).

16. The system of claim 9, wherein the secretory protein is secetory alkaline phosphatase (SEAP).


SEQUENCE LISTING (8 sequences)

SEQ ID NO: 1 - DNA, 60 bp, Perina nuda picorna like virus
gcccaggggtgggttccggatttgactgtggatggagatgttgagtcaaatcccggaccc

SEQ ID NO: 2 - protein, 20 aa, Perina nuda picorna like virus
AQGWVPDLTVDGDVESNPGP

SEQ ID NO: 3 - DNA, 60 bp, Perina nuda picorna like virus
attggtggtgggcagaaggatttgacacaagatggtgacatcgagtcgaatcctgggccc

SEQ ID NO: 4 - protein, 20 aa, Perina nuda picorna like virus
IGGGQKDLTQDGDIESNPGP

SEQ ID NO: 5 - DNA, 41 bp, ARTIFICIAL SEQUENCE
  Forward primer for fusion construct
atataagatctccaccatgctgctgctgtgctgctgctggg

SEQ ID NO: 6 - DNA, 34 bp, ARTIFICIAL SEQUENCE
  Reverse primer for fusion construct
aattcagatctggtgtctgctcgaagcggccggc

SEQ ID NO: 7 - DNA, 38 bp, ARTIFICIAL SEQUENCE
  Forward primer for fusion construct
aatggatccgctagcccaccatgctgctgctgctgctg

SEQ ID NO: 8 - DNA, 90 bp, ARTIFICIAL SEQUENCE
  Reverse primer for fusion construct
aatagatctaagggtccggggatttgactcaacatctccatccacagtcaaatccggaacccacccctgggctgtctgctcgaagcggcc